(19) United States
(12) Patent Application Publication
(10) Pub. No.: US 2014/0249389 A1
(43) Pub. Date: Sep. 4, 2014

(54) METHOD AND APPARATUS FOR TREATING SKIN PRIOR TO BIOSENSOR INSERTION

(71) Applicant: iSense Acquisition, LLC, Wilsonville, OR (US)

(72) Inventors: William Kenneth Ward, Portland, OR (US); Robert Bruce, Beaverton, OR (US); Les Wood, Wilsonville, OR (US); Gary Lichttenegger, Woodinville, WA (US)

(73) Assignee: iSense Acquisition, LLC, Wilsonville, OR (US)

(21) Appl. No.: 14/280,330

(22) Filed: May 16, 2014

Related U.S. Application Data
(62) Division of application No. 12/238,260, filed on Sep. 25, 2008.
(60) Provisional application No. 60/975,077, filed on Sep. 25, 2007.

(51) Int. Cl. A61B 5/145

(52) U.S. Cl. CPC A61B 5/145
USPC 600/309

(57) ABSTRACT

Embodiments of the present invention provide a device and method for treating animal skin to produce tautness in the skin during the insertion of an analyte sensor. A device in accordance with embodiments of the present invention includes a housing that partially encloses a cavity and is adapted for mounting onto animal skin and a sensor insertion module adapted to insert an analyte sensor through animal skin.

### CROSS REFERENCE TO RELATED APPLICATIONS

The present application claims priority to U.S. patent application Ser. No. 12/238,260, filed Sep. 25, 2008, entitled “Method and Apparatus for Treating Skin Prior to Biosensor Insertion,” which claims priority to U.S. Provisional Patent Application No. 60/975,077, filed Sep. 25, 2007, entitled “Method and Apparatus for Treating Skin Prior to Biosensor Insertion,” the entire disclosures of which are hereby incorporated by reference in their entirety.

### TECHNICAL FIELD

Embodiments of the present invention relate to the field of medical devices, and, more specifically, to methods and apparatuses for tautening animal skin in preparation for the insertion of an analyte sensor.

### BACKGROUND

Electrochemical sensors are used to measure chemical fluctuations in body fluids or other materials. For example, glucose levels may be closely monitored by implanting a subcutaneous electrochemical sensor that measures glucose in interstitial fluid. An electrochemical sensor may be fabricated onto one end of a thin wire which is then inserted through the dermis, placing the sensor portion in subcutaneous tissue while the other end of the wire remains outside the body. The sensor is electrically connected to a reference electrode and to an external electronic device that interprets electrical current from the sensor to display a glucose value. Once implanted, the sensor monitors glucose levels continuously and may remain in place for five to seven days, offering improved convenience and consistency for users who must self-monitor blood glucose levels.

The insertion of an analyte sensor through the skin of the user and into subcutaneous tissue can be accomplished with the use of an external sharpened needle (trocar) or a hard internal element (stylet). A more user-friendly approach is auto-injection/auto-insertion, in which the analyte sensor itself is used to pierce the skin; this eliminates the need for a stylet or trocar during the insertion process and reduces the risk of injury associated with sharp metal parts. Thinner and more flexible sensors also minimize skin disruption and user discomfort. However, the reduction in the size and mass of the sensor increases the motive force required to insert the sensor through the dermal layers.

One difficulty in the insertion of analyte sensors is that they may be difficult to insert through loose skin, such as the skin of the abdomen. During insertion, motive force is applied to an analyte sensor, causing the sensor to pierce the stratum corneum and the underlying epidermal layers and resulting in the placement of the sensor tip in the subcutaneous tissues of the user. Loose skin is problematic for sensor insertion because it disperses force with much greater efficiency than taut skin, making loose skin more difficult to pierce. This effect is analogous to the dispersion of a soccer ball's forward inertia by a slack soccer net, which reduces the force exerted by the ball at the point of impact by dispersing the force over a wider area. Attempted insertion of a thin and flexible sensor into flaccid skin may result in sensor deflection, bending or kinking, and/or failed insertion, presenting a significant challenge for users of subcutaneous analyte sensors.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1 illustrates a cross-sectional view of a sensor module and skin tautening device in accordance with various embodiments of the present invention;

FIG. 2 illustrates a bottom perspective view of a skin tautening device in accordance with various embodiments of the present invention;

FIG. 3A illustrates a perspective view of a skin tautening device in accordance with various embodiments of the present invention;

FIG. 3B illustrates perspective views of elastomeric modules for use with a skin tautening device in accordance with various embodiments of the present invention;

FIG. 3C illustrates a perspective view of a skin tautening device in accordance with various embodiments of the present invention; and

FIG. 4 illustrates a cross-sectional schematic view of a sensor module and skin tautening device in accordance with embodiments of the present invention.

### DETAILED DESCRIPTION OF EMBODIMENTS OF THE INVENTION

In the following detailed description, reference is made to the accompanying drawings which form a part hereof, and in which are shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments herein.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

For the purposes of the description, a phrase in the form “NB” or in the form “A and/or B” means “(A), (B), or (A and B)”. For the purposes of the description, a phrase in the form “at least one of A, B, and C” means “(A), (B), (C), (A and B), (A and C), (B and C), or (A, B and C)”. For the purposes of the description, a phrase in the form “(A)B” means “(B) or (AB)” that is, A is an optional element.

For the purpose of describing embodiments of the present invention, the term “mounted,” along with its derivatives, may be used. The term “mounted” may be used to indicate that one or more elements are in direct physical contact with another element, or that one or more elements are in direct physical contact with animal (including human) skin. The term “mounted” may also be used to indicate that two or more elements, or one or more elements in addition to animal skin, are not in direct contact with each other, but yet still cooperate or interact with each other either directly or indirectly, such as through one or more other elements, such as an elastomeric seal, a liquid, an adhesive and/or gelatinous substance, etc.

For the purpose of describing embodiments of the present invention, the terms “auto-insertion” and “auto-inserter,” along with their derivatives, may be used. The term “auto-insertion” may be used to indicate the insertion of an analyte sensor through animal skin using the sensor itself to pierce the skin. The term “auto-inserter” may be used as a term for a device that inserts an analyte sensor through animal skin using the sensor itself to pierce the skin.

In various embodiments of the present invention, methods and apparatuses for tautening animal skin in preparation for the subcutaneous or transcutaneous insertion of an analyte sensor are provided. In an embodiment, there is provided an animal skin tautening device comprising a housing which partially encloses a cavity and is adapted for mounting onto the skin of an animal, such as a human, and a sensor insertion module adapted to accommodate at least a portion of an analyte sensor. In embodiments, a sensor insertion module may further include a guidance structure adapted to provide axial support to an analyte sensor, an exit port, electronic circuitry, and/or an insertion activation device which may further include a mechanism to provide high speed motive force to the analyte sensor.

In an embodiment of this invention, a sensor insertion module may be used in association with a hollow chamber/cavity open at least partially at its bottom end. In an embodiment, a hollow open-ended chamber/cavity may be placed onto animal skin such that the open-ended bottom surface of the hollow chamber/cavity is in physical contact with the animal skin or is in close proximity to the animal skin. In an embodiment, pressure within the hollow chamber/cavity may be reduced, pulling animal skin against the open end of the chamber/cavity to seal the opening. In an embodiment, animal skin within the perimeter of the bottom surface of the hollow chamber/cavity may be pulled upward toward the interior of the hollow chamber/cavity by the negative pressure, thus tautening the skin. In an embodiment, motive force may then be provided through a sensor insertion module to insert an analyte sensor through the tautened animal skin.

Thus, as provided in an embodiment, a skin tautening device may include a housing configured for mounting onto animal skin, the housing having an outer surface, a proximal end and a distal end, and an inner surface, the inner surface defining a cavity with an opening and adapted for the induction of negative pressure, wherein placement of the opening against the animal skin and induction of negative pressure within the cavity pulls animal skin at least partially into the cavity. An embodiment may also include a sensor insertion module coupled to the housing and projecting at least partially through the housing, wherein the sensor insertion module is adapted to insert an analyte sensor into subcutaneous animal tissue. In some embodiments, a sensor insertion module may include a trocar and/or a stylet for sensor insertion, while in other embodiments, a sensor insertion module may lack a trocar and/or a stylet.

In an embodiment of this invention, a sensor insertion module may be used in association with a hollow chamber open at the bottom end, which may be placed onto animal skin such that the open bottom surface of the chamber is placed against the skin. In an embodiment, the sides of the open-ended hollow chamber may be constructed at least partially of an elastomeric material and may include one or more elastomeric components arranged around at least a portion of the perimeter of the device. In an embodiment, elastomeric components arranged around the device may form finger-like projections which are substantially perpendicular to the animal skin prior to insertion of the sensor. In an embodiment, elastomeric components may include textural features such as ridges, grooves, smaller finger-like projections, etc. In an embodiment, an elastomeric component may be adapted to respond to force applied down the length of the component by bending and/or splaying outward at its distal edge, which may be in direct physical contact with animal skin. In an embodiment, a component that bends and/or splays outward may apply lateral force to the animal skin, stretching the skin and/or causing the skin to become taut. In an embodiment, motive force may be provided through a sensor insertion module to insert a sensor through tautened animal skin.

Thus, as provided in an embodiment, a skin tautening device may include a housing configured for mounting onto animal skin, the housing having an outer surface, a proximal end and a distal end, and/or one or more elastomeric components, wherein placement of the housing against the animal skin and application of force to the housing in a direction toward the animal skin exerts lateral force on the animal skin. For the purposes of describing embodiments herein, the phrase “in a direction toward animal skin” refers broadly to any direction toward the skin on which a housing/module is being applied whether perpendicular to the skin or at an angle. An embodiment may also include a sensor insertion module coupled to the housing and projecting at least partly through the housing, wherein the sensor insertion module is adapted to insert an analyte sensor into subcutaneous animal tissue. In some embodiments, a sensor insertion module may include a trocar and/or a stylet for sensor insertion, while other embodiments may not include a trocar and/or a stylet.

In an embodiment of this invention, a sensor insertion module may be used in association with a guidance structure that extends partially outside the body of the sensor insertion module. In such an embodiment, the extended portion of the guidance structure may cause dimpling of the skin thus tautening the skin prior to insertion of the sensor.

Thus, as provided in an embodiment, a skin tautening device may include a housing configured for mounting onto animal skin, the housing having an outer surface, a proximal end and a distal end, and a guidance structure extending at least partially out of the housing and providing a structure for directional guidance of a sensor through the housing and into animal skin, wherein placement of the housing against the animal skin and application of force to the housing exerts force on the animal skin to tauten the animal skin, and a sensor insertion module coupled to the housing and projecting at least partly through the housing, wherein the sensor insertion module is adapted to insert an analyte sensor into animal tissue.

In some embodiments a skin tautening device may be used to insert an analyte sensor through skin that is free of scar tissue, while in other embodiments a skin tautening device may be used to insert an analyte sensor through animal skin in a location where scar tissue is present. In an embodiment, an animal and/or its skin may be positioned to minimize or prevent folding of skin at or near the site of sensor insertion prior to the application of a skin tautening device. In an embodiment of the present invention, a skin tautening device may be used to insert an analyte sensor through the abdominal skin of a human. In some embodiments, a human may be standing during the application of the skin tautening device and/or sensor insertion, while in other embodiments, a human may be sitting upright such as while leaning back at the waist to minimize abdominal skin folding, leaning forward, lying down, etc. In other embodiments, a skin tautening device may be used to insert an analyte sensor through the skin of a human in other locations, such as through the skin of the back, arms, legs, etc.

As provided in an embodiment, animal skin may be tautened prior to analyte sensor insertion by providing a skin tautening device that is adapted for mounting onto animal skin and includes a sensor insertion module, placing a tautening device against animal skin, actuating a tautening device, wherein actuation of a tautening device tautens animal skin, and actuating a sensor insertion module. In some embodiments, actuation of a sensor insertion module may result in the insertion of an analyte sensor through animal skin. In an embodiment, actuation of a tautening device may simply be provided by manual pressure, such as by pressing down on a module, for example to tension the skin, for example by dimpling.

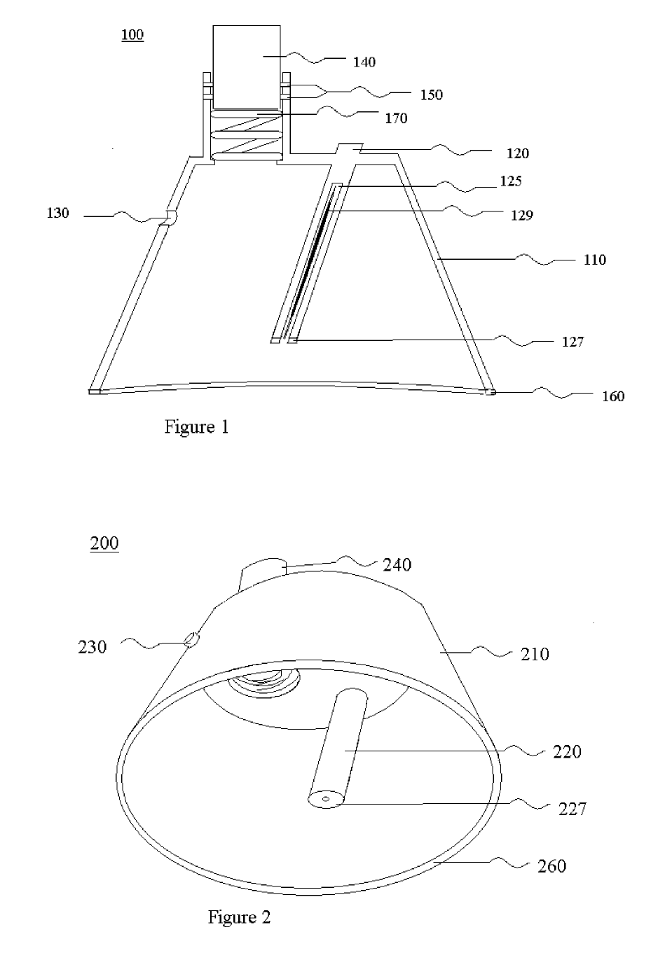

FIG. 1 is a cross-sectional illustration of an embodiment of the present invention in which a sensor module and skin tautening device 100 includes a housing 110, a sensor insertion module 120 at least partially enclosed by housing 110, a valve 130 at least partially enclosed along its outer perimeter by housing 110, a plunger 140 partially enclosed by housing 110, a plunger seal 150 at least partially enclosed by housing 110, an elastomeric vacuum seal 160 reversibly affixed to housing 110, and a spring 170 at least partially enclosed by housing 110. In an embodiment, a sensor insertion module 120 may further include a guidance structure 125 circumferentially enclosed by housing 110, a pressure-sensitive actuator tip 127 mechanically connected to guidance structure 125 and/or an analyte sensor 129 at least partially enclosed by guidance structure 125.

In the operation of an embodiment, plunger 140 may be depressed and subsequently maintained in a depressed position while housing 110 is placed in physical contact against animal skin such that the bottom surface of elastomeric vacuum seal 160 is in physical contact with animal skin. In an embodiment, plunger 140 may then be released, creating negative pressure within the cavity of housing 110. In an embodiment, negative pressure within the cavity of housing 110 may cause animal skin within the circumference of elastomeric vacuum seal 160 to be drawn upward until physical contact is made between animal skin and pressure-sensitive actuator tip 127. In an embodiment, physical contact between animal skin and pressure-sensitive actuator tip 127 may cause sensor insertion module 120 to provide sufficient motive force to inject/insert analyte sensor 129 through tautened animal skin.

In an alternative embodiment, plunger 140 may be depressed after housing 110 is placed on animal skin such that the bottom surface of elastomeric vacuum seal 160 is in physical contact with animal skin. In an embodiment, subsequent depression of plunger 140 may force displaced air through valve 130, for example a one-way valve. In an embodiment, plunger 140 may then be released, creating negative pressure within the cavity of housing 110 and causing animal skin within the perimeter of the bottom of elastomeric vacuum seal 160 to be drawn upward and tautened until physical contact is made between animal skin and pressure-sensitive actuator tip 127. In an embodiment, pressure from tautened animal skin on pressure-sensitive actuator tip 127 may trigger the injection/insertion of analyte sensor 120 through animal skin by sensor insertion module 120.

In an embodiment, housing 110 may be bell-shaped. However, in other embodiments, housing 110 may have other shapes; for example, housing 110 may be shaped as a cylinder, a hemisphere, a cone, a pyramid, etc. In some embodiments, housing 110 may be constructed as a single piece, while in other embodiments housing 110 may be composed of a multiplicity of units which may vary in size, shape and composition. The material composition of housing 110 may vary among different embodiments but may include any rigid, semi-rigid or flexible material, such as glass, rubber, ceramics, metals, plastics, composites, resins, polymers, etc., and may vary in opacity and in other physical characteristics.

In an embodiment, sensor insertion module 120 may not be removable from housing 110, while in other embodiments sensor insertion module 120 may be reversibly coupled to housing 110. In an embodiment, a sensor insertion module may include a stylet and/or a trocar for sensor insertion. In some embodiments, a sensor insertion module may include a source of high speed motive force for the insertion of an analyte sensor through animal skin, whether via an integrated device or a separately coupled device. In other embodiments, motive force for insertion may be supplied manually, for example, by manually pushing a stylet through the skin, by manually pushing a trocar through the skin, by manually pushing an analyte sensor through the skin, etc. Some embodiments may include one or more sources of motive force for the insertion of an analyte sensor through animal skin. In an embodiment, insertion of an analyte sensor may be aided by breaching animal skin with a breaching aid such as a gas, a liquid, and/or a solid, for example a trocar, stylet, hypodermic needle, high-pressure liquid, high-pressure gas, etc., prior to analyte sensor insertion.

In an embodiment, a sensor insertion module may further include a guidance structure adapted to provide axial support to an analyte sensor, an exit port, an insertion activation device further including a mechanism to provide high speed motive force to the analyte sensor, and/or electronic circuitry. Although the source of the high speed motive force may vary among embodiments that include a source of high speed motive force, a high speed motive force may be provided by devices such as a solenoid, a spring, a CO₂ or other compressed gas cartridge, an air pump, a structure adapted to maintain a sensor in a bowed configuration such that the sensor holds potential energy, chemical reactions, an electromagnetic accelerator, a pyrotechnic charge, etc. Additional details about suitable devices for actuation of a sensor insertion module may be found in U.S. patent application Ser. No. 11/558,394, filed Nov. 9, 2006, the entire contents of which are hereby incorporated by reference, which describes a method and apparatus for insertion of an analyte sensor without an introducer.

In an embodiment, application of pressure to pressure-sensitive actuator tip 127 may trigger the insertion of analyte sensor 129 through tautened animal skin. Some embodiments may lack pressure-sensitive actuator tip 127.

In an embodiment, other stimulus-sensitive actuators may be provided. In an embodiment, a chemical-sensitive actuator tip may trigger the injection/insertion of analyte sensor 129 in response to contact with one or more elements and/or chemicals on animal skin. In another embodiment, a heat-sensitive actuator tip may trigger the insertion of analyte sensor 129 upon contact with animal skin. In other embodiments, an actuator tip sensitive to variations in light, pH, moisture, or electrical conductivity may trigger the insertion of analyte sensor 129 upon contact with animal skin.

While stimulus-sensitive actuators have been described as being located on a guidance structure, in embodiment, stimulus-sensitive actuators may be located on other portions of a housing/module.

The insertion of analyte sensor 129 through animal skin may be triggered manually in some embodiments, such as by operating a button, switch, touchpad, etc. In other embodiments, the insertion of an analyte sensor may be controlled by a timer.

In some embodiments, motive force for the extension/retraction of a stylet and/or trocar may be provided manually. In other embodiments, motive force for the extension/retraction of a stylet and/or trocar may be provided by a solenoid, a spring, a CO₂ or other compressed gas cartridge, an air pump, a structure adapted to maintain a sensor in a bowed configuration such that the sensor holds potential energy, chemical reactions, an electromagnetic accelerator, a pyrotechnic charge, etc. In an embodiment, the extension/retraction of a stylet and/or trocar may be triggered manually, such as by operating a button, switch, touchpad, etc. In other embodiments, the extension/retraction of a stylet and/or trocar may be triggered by negative pressure, one or more chemicals, heat, friction, light, pH, moisture, electrical conductivity, a timer, etc. An embodiment may include a combination of two or more sources of motive force and/or trigger mechanisms.

In an embodiment, a skin tautening device may include one or more sensor insertion modules. In an embodiment, a sensor insertion module may accommodate and/or insert one or more analyte sensors. In some embodiments, a sensor insertion module may insert materials for use with an analyte sensor such as an antimicrobial drug/device, an electronic component, a membrane, etc. through animal skin.

In an embodiment, valve 130 may be a unidirectional valve and may be at least partially enclosed by housing 110. In an embodiment, valve 130 may allow air to flow from the cavity of housing 110 to the exterior but not in the reverse direction (i.e. into the cavity). In an alternative embodiment, valve 130 may be adapted to be adjustable to regulate air flow between the cavity of housing 110 and the exterior of skin tautening device 100. Some embodiments may lack a valve while other embodiments may include one or more valves. An embodiment may include one or more valve types, such as a solenoid valve, a gate valve, an inline valve, an angle valve, a ball valve, a butterfly valve, a conductance controller valve, a diaphragm valve, a spring loaded valve, etc. In an embodiment, one or more valves may be adapted to be mechanically coupled to a device and/or to a hose/tube coupled to a device used for the removal of air from a cavity of housing 110, such as a manual or motor-driven vacuum pump, a household vacuum cleaner, an elastomeric bulb, a syringe, a bellows, etc. In an embodiment, housing 110 may include a heating coil on or near the upper surface of the cavity of housing 110 to increase air temperature within the cavity of housing 110, forcing air through valve 130 and producing negative pressure upon cooling of the remaining air within the cavity. In an embodiment, valve 130 may be adapted for the application of oral suction and/or may be mechanically coupled to a hose/tube adapted for oral suction. Some embodiments may include one or more elements adapted to regulate and/or disperse negative pressure within the cavity of housing 110, such as a pressure gauge, a membrane, a pressure-sensitive valve, a vent, etc.

In an embodiment, spring 170 may exert upward force on plunger 140 to increase the cavity volume of housing 110, while two plunger seals 150 may prevent leakage of air into the cavity of housing 110 around plunger 140. An embodiment may include only one plunger seal 150 or two, three, or a multiplicity of plunger seals 150. In other embodiments, motive force to push plunger 140 upward may be provided by manually pulling plunger 140 or by manually manipulating a button, wheel, lever or other control device. In an embodiment, the exterior surface of plunger 140 may be modified to include spiraling threads which allow plunger 140 to be depressed and returned upright by turning plunger 140. In other embodiments, motive force to push plunger 140 upward may be provided electronically and/or by a motor. In an embodiment, motive force to push plunger 140 upward may be provided by an elastomeric structure such as a membrane, a net, a band, etc. Some embodiments may not include a plunger.

In an embodiment, elastomeric vacuum seal 160 may extend continuously along the bottom rim of housing 110 and may be composed at least in part of an elastomer, such as rubber, nitrile, propylene, silicone, polyurethane, chloroprene, fluorocarbon, fluorosilicone, liquid silicone rubber, etc. Other embodiments may lack elastomeric vacuum seal 160. In an embodiment, a soft solid and/or viscous liquid, such as petroleum jelly, oil, gelatin, liquid adhesive, gel adhesive, etc., may be applied to the bottom rim of housing 110 and/or to animal skin prior to mounting skin tautening device 100 on animal skin in order to create a seal between them. In an embodiment, housing 110 may be composed of an elastomeric material and may lack an elastomeric vacuum seal.

In embodiments, housing 110 may include a mechanism adapted for venting the cavity of housing 110 such as a port, valve, vent, etc.

FIG. 2 shows a perspective view of skin tautening device 200. In an embodiment, plunger 240 may be depressed and maintained in that position, and housing 210 may be subsequently mounted onto animal skin such that elastomeric seal 260 is in physical contact with animal skin along the complete perimeter of the bottom of elastomeric seal 260. In an embodiment, plunger 240 may then be released to create negative pressure within the cavity of housing 210, tautening animal skin within the perimeter of elastomeric seal 260 and pulling the skin upward toward pressure-sensitive actuator tip 227. In an embodiment, pressure exerted by tautened animal skin on pressure-sensitive actuator tip 227 of sensor insertion module 220 may trigger the insertion of an analyte sensor through tautened animal skin. In an embodiment, valve 230 may be adapted to regulate air flow between the cavity of housing 210 and the exterior of skin tautening device 200. In an embodiment, valve 230 may be provided to allow air to flow from the cavity of housing 210 to the exterior but not in the reverse direction (i.e. into the cavity). In other embodiments, the shapes and dimensions of housing 210 and of the cavity partially enclosed by housing 210 may vary. In an embodiment, the sensitivity of pressure-sensitive actuator tip 227 may be adjustable.

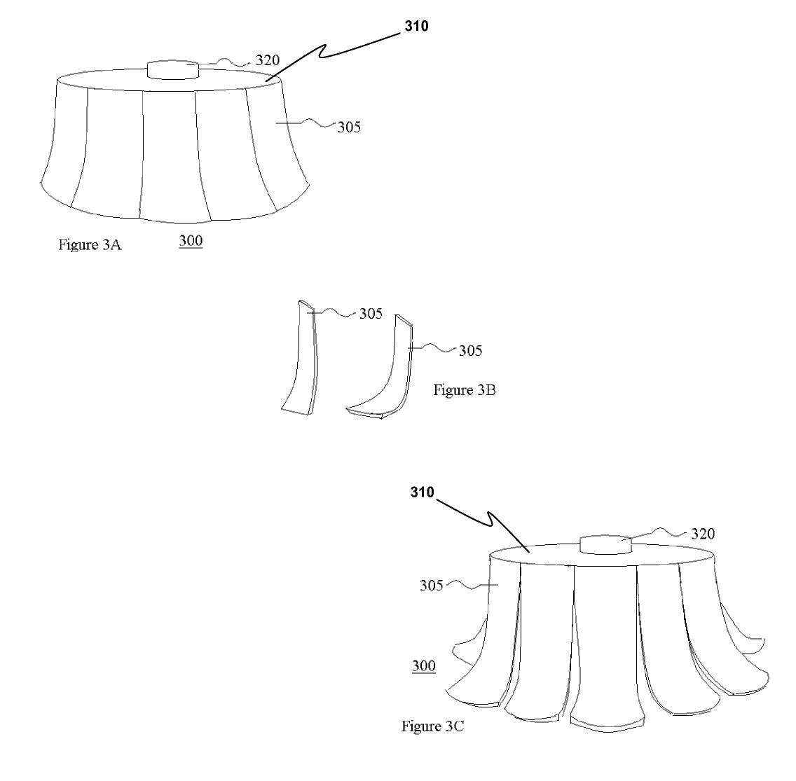

FIGS. 3A, 3B and 3C illustrate embodiments and components of a skin tautening device 300 adapted to displace downward force in a direction substantially perpendicular to the force applied, using lateral mechanical force instead of negative pressure to tauten animal skin. In an embodiment, housing 310 may be constructed of a multiplicity of elastomeric modules 305 connected at their upper edges and arranged in a bell shape surrounding sensor insertion module 320, as shown in FIG. 3A. In an embodiment, one or more elastomeric modules 305 may be adapted to grip animal skin at its bottom edge and to displace downward force in a direction perpendicular to the force applied, as shown in FIG. 3B. Downward force may be applied to the top of skin tautening device 300 causing an elastomeric module 305 to splay outward, exerting lateral force directed away from the center of the device on animal skin gripped by the bottom edge of an elastomeric module 305, as shown in FIG. 3C.

In the operation of this embodiment, housing 310 may be placed on animal skin such that the bottom surface of an elastomeric module 305 along the perimeter of housing 310 is in physical contact with animal skin. In an embodiment, downward force (such as perpendicular to animal skin) may then be applied to the top of housing 310, causing one or more elastomeric modules 305 to splay outward, tautening the animal skin within the perimeter of the device. An analyte sensor may then be inserted through the tautened animal skin by sensor insertion module 320.

In some embodiments, insertion of an analyte sensor may be triggered by the pressure exerted by tautened animal skin on a pressure-sensitive actuator tip. Other embodiments may not include an actuator tip. In some embodiments, the insertion of an analyte sensor may be triggered manually, such as by operating a button, switch, touchpad, etc., and/or by an actuator sensitive to other stimuli such as heat, pH, a chemical, electrical conductivity, moisture, light, etc.

In an embodiment, one or more elastomeric modules 305 may include a longitudinal rod to prevent undesirable excessive splaying and/or bending. In other embodiments, undesirable excessive splaying and/or bending may be prevented by the addition of one or more annular latitudinal elements to the interior and/or exterior of the skin tautening device. In an embodiment, excessive splaying and/or bending may be prevented by other mechanisms which may include but are not limited to the addition of one or more springs, bands, longitudinal plates, etc. In an embodiment, one or more additional housing structures may be used on the exterior and/or the interior of a skin tautening device to prevent excessive splaying and/or bending.

In some embodiments, each elastomeric unit 305 may be mechanically coupled to its neighboring elastomeric unit 305 at its top or side, while in other embodiments, elastomeric units 305 may not be mechanically coupled to other elastomeric units 305. An embodiment of a skin tautening device may include a single hollow elastomeric unit partially enclosing a sensor insertion module. In an embodiment, a single hollow elastomeric unit may be vertically divided into sections such that the lower portion of the unit has one or more vertical finger-like projections which are not wholly detached from the single elastomeric unit.

Some embodiments may combine negative pressure and lateral pressure for skin tautening. In an embodiment, a housing may include one or more concentric and/or nested open-ended chambers. In an embodiment, the perimeter of the outermost open-ended chamber may at least partially enclose the perimeter of the innermost open-ended chamber. In an embodiment, negative pressure may be induced in one or more open-ended chambers. In an embodiment, one or more open-ended chambers may include elastomeric units adapted to apply lateral pressure to animal skin. In an embodiment, lateral pressure may be applied by one or more elastomeric units prior to the induction of negative pressure. In other embodiments, negative pressure may be induced prior to application of lateral pressure on animal skin by elastomeric units. In some embodiments, the induction of negative pressure and the application of lateral force on animal skin may occur simultaneously.

Other embodiments may cause tautening of animal skin by application of downward pressure and/or by compression of animal skin. In an embodiment, compressed gasses and/or a pneumatic device may exert downward force on animal skin prior to and/or during insertion of an analyte sensor. In other embodiments, a hollow open-ended rod surrounding sensor insertion module 320 may be pressed against animal skin to tauten the skin. In an embodiment, sensor insertion module 320 may then be manually or automatically triggered to insert one or more analyte sensors.

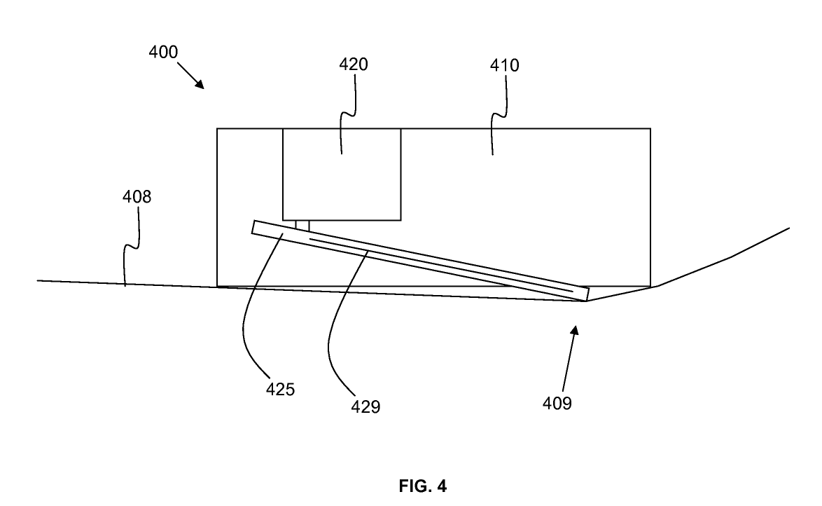

FIG. 4 is a cross-sectional illustration of an embodiment of the present invention in which a sensor module and skin tautening device 400 includes a housing 410, a sensor insertion module 420 at least partially enclosed by housing 410. In an embodiment, a sensor insertion module 420 may further include a guidance structure 425 circumferentially enclosed by housing 410 and an analyte sensor 429 at least partially enclosed by guidance structure 425. In the operation of an embodiment, housing 410 may be depressed against skin 408 forcing an extended (distal) portion of guidance structure 425 to dimple skin 408 at region 409 to cause tensioning of skin 408 prior to insertion of sensor 429 into skin 408.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A skin tautening device, comprising:
a housing configured for mounting onto animal skin, the housing having an outer surface, a proximal end, and a distal end, and one or more elastomeric components, wherein placement of the housing against the animal skin and application of force to the housing exerts lateral force on the animal skin to tauten the animal skin; and
a sensor insertion module coupled to the housing and projecting at least partly through the housing, wherein the sensor insertion module is adapted to insert an analyte sensor into animal tissue.

2. The skin tautening device of claim 1, further comprising a breaching aid coupled to or provided by the sensor insertion module.

3. The skin tautening device of claim 2, wherein the breaching aid comprises a stylet.

4. The skin tautening device of claim 2, wherein the breaching aid comprises a trocar.

5. The skin tautening device of claim 2, wherein the breaching aid comprises a liquid adapted for delivery under high-pressure.

6. The skin tautening device of claim 2, wherein the breaching aid comprises a gas adapted for delivery under high-pressure.

7. The skin tautening device of claim 1, further comprising an auto-inserter coupled to the sensor insertion module.

8. The skin tautening device of claim 7, wherein the auto-inserter includes a high-speed motive force device.

9. The skin-tautening device of claim 8, wherein the high-speed motive force device comprises a solenoid.

10. The skin tautening device of claim 8, wherein the high-speed motive force device comprises a spring.

11. The skin tautening device of claim 8, wherein the high-speed motive force device comprises a compressed gas cartridge.

12. The skin tautening device of claim 8, wherein the high-speed motive force device comprises an air pump.

13. The skin tautening device of claim 8, wherein the high-speed motive force device comprises an electromagnetic accelerator.

14. The skin tautening device of claim 1, further comprising a guidance structure coupled to the sensor insertion module for providing axial support to a sensor.

15. The skin tautening device of claim 1, further comprising a stimulus-sensitive actuator configured to facilitate movement of a sensor when actuated.

16. The skin tautening device of claim 15, further comprising a guidance structure coupled to the sensor insertion module for providing axial support to a sensor, wherein the stimulus-sensitive actuator is located on the guidance structure.

17. The skin tautening device of claim 15, wherein the stimulus-sensitive actuator comprises an actuator sensitive and responsive to at least one of pressure, one or more chemicals, heat, light, pH, moisture, or electrical conductivity.
